(19) United States
(12) Patent Application Publication
(10) Pub. No.: US 2005/0159439 A1
(43) Pub. Date: Jul. 21, 2005

(54) Management of postoperative pain

(76) Inventors: Marco Pappagallo, New York, NY (US); Luciano Mastronardi, Rome (IT)

Correspondence Address: PATREA L. PABST;PABST PATENT GROUP LLP, 400 COLONY SQUARE, SUITE 1200, ATLANTA, GA, 30361, US

(21) Appl. No.: 10/496,451

(22) PCT Filed: Oct. 24, 2002
(86) PCT No.: PCT/US02/34161

Related U.S. Application Data
(60) Provisional application No. 60/348,016, filed on Oct. 24, 2001.

(51) Int. Cl. A61K 38/18; A61K 31/485; A61K 9/14

(52) U.S. Cl. 514/282; 514/2; 424/489

(57) ABSTRACT

A formulation containing an opioid such as morphine in a low dose in a controlled release carrier, such as the carbohydrate polymer gel marketed as ADCON®*-L, can be administered to patients to treat pain over a period of one or more days following surgery of the spinal column or other structures associated with the central nervous system. In one embodiment, the morphine is administered at the end of a lumbar microdicectomy. A low dose can be used as compared to previous studies in which the morphine was administered in solution, typically at levels of about 2-4 mg morphine when administered in a paste, or 10 mg morphine when administered as an epidural solution.

### BACKGROUND OF THE INVENTION

The present invention is generally in the field of pain control, and specifically relates to a method of treating pain following spinal column surgery using a controlled release formulation of an opioid such as morphine.

Lumbar disc herniation is the most common spinal disorder. (Apostolides P J, et al., Lumbar discectomy Microdiscectomy: “The Gold Standard”, Clin Neurosurg 43: 228-238 (1995)) Microdiscectomy is the “gold standard” of surgical therapy for uncomplicated disk herniations. Microdiscectomy is performed in symptomatic patients whose disabling pain and functional impairment have failed to respond to adequate trials of conservative treatments. (Apostolides, et al.) However, in the first few postoperative days, the surgical pain and stress derived from this minimally invasive procedure can cause further discomfort. For this reason, many patients are unable to sustain physical mobilization in the immediate postoperative period. Unnecessarily prolonged hospitalization and repeated administration of parenteral analgesics, with increased potential for unwanted drug related side-effects, often follows.

The postoperative pain described above is a typical nociceptive pain. It is initiated and maintained by the chemical mediators of tissue inflammation.

Opioid analgesics are a known and successful treatment of nociceptive pain. They achieve a direct analgesic effect by targeting the mu, kappa, and delta opiate receptors, which are located at different levels along the nociceptive pathways. (Pasternack G W “Progress in opiate pharmacology”, in Chapmann C R, Foley K M (eds). Current and Emerging Issues in Cancer Pain: Research and Practice, New York, Raven Press, pp. 247-287 (1993); Zhang Q, et al, “Effects of neurotoxins and hind paw inflammation on opioid receptor immunoreactivities in dorsal root ganglia”, Neuroscience 85: 281-91 (1998)) Opioids are believed to act primarily at the spinal cord level, on the incoming nociceptor endings within the dorsal horn. (Stein C, et al., “Peripheral opioid receptors mediating antinociception in inflammation. Evidence for involvement of mu, delta and kappa receptors”, J Pharmacol Exp Ther 248: 1269-1275 (1989); Stein C, et al., “Analgesic effect of intraarticular morphine after arthroscopic knee surgery”, N Engl J Med 325: 1123-126 (1991))

Evidence gathered from animal studies and clinical observations indicates that opioids also act on the nociceptor endings within the inflamed peripheral tissues. Bryant C J, et al., “Use of intra-articular morphine for postoperative analgesia following TMJ arthroscopy”, Br J Oral Maxillofac Surg 37: 391-361 (1999); Machelska H, et al., “Peripheral effects of the kappa-opioid agonist EMD 61753 on pain and inflammation in rats and humans”, J Pharmacol Exp Ther 290: 354361 (1999); Stein, J Pharmacol Exp Ther; Stein, N Engl J Med) In fact, several studies have demonstrated not only the presence of opioid inducible receptors in small nerve fibers innervating the site of injury, but also the peripheral analgesic effects of opioids. (Machelska; Stein, J Pharmacol Exp Ther; Stein, N Engl J Med; Zhang, Neuroscience; Zhou L, et al., “Contribution of opioid receptors on primary afferent versus sympathetic neurons to peripheral opioid analgesia”, J Pharmacol Exp Ther 286:1000-1006 (1998))

The nociceptive sensory neurons of the dorsal root ganglia synthesize the opioid receptor components, which are then transported along their proximal and distal axons towards the central and peripheral nerve endings. Activation of such receptors results in an antinociceptive effect that is most prominent in pain secondary to tissue inflammation. (Zhou, J Pharmacol Exp Ther) Zhang et al. examined the regulation of opioid receptors in dorsal root ganglia of rats after hind paw induced inflammation. (Zhang, Neuroscience) The authors observed that peripheral inflammation differentially regulates the expression of opioid receptors in dorsal root ganglia neurons, with an up-regulation of mu-receptors and down-regulation of delta- and kappa-receptors. Morphine, a potent mu opioid agonist, represents the mainstay for the treatment of moderate to severe nociceptive pain. (Pappagallo M, “Aggressive pharmacologic treatment of pain”, in Pisetsky D S, Bradley L (eds): Pain Management in the Rheumatic Diseases. Rheumatic Dis Clin North Am 1: 193-213 (1999); Portenoy R K, “Opioid therapy for chronic nonmalignant pain: Current status”, in Fields H L, Liebeskind J C (eds): Progress in Pain Research and Management. Seattle, IASP Press, pp 219-247 (1994)).

Opioids are the most effective analgesics in the treatment of acute and chronic pain. (Moulin D E, et al. “Randomized trial of oral morphine for chronic non-cancer pain” Lancet 347: 143-147 (1996); Needham C W. “Painless lumbar surgery: morphine nerve paste”, Conn Med 60: 141-143 (1996); and Pappagallo M. “Aggressive pharmacologic treatment of pain” In Pisetsky D S, Bradley L (eds): Pain Management in the Rheumatic Diseases. Rheumatic Dis Clin North Am 1: 193-213 (1999)) Modem commentary suggests that the therapeutic use of morphine is as frequent as the consumption of chicken (see e.g. Damien Hirst's print entitled “The Last Supper” Museum Of Modem Art, New York, N.Y., screen print no. 9(1999)); however, the medical use of effective analgesics such as opioids for severe pain is still under-utilized in many countries. According to the American Medical Association's Council of Scientific Affairs, health care professionals continue to undertreat patients due to the fear of some opioid-related side effects, such as respiratory depression, and issues such as addiction, tolerance, and physical dependence. (Joint Commission on Accreditation of Healthcare Organization. “Pain Management Today”, In Pain Assessment and Management. An Organizational Approach. Chapt 1, pp. 1-6 (2000) (herein referred to as “Joint Commission”)) In addition to the fear of administering opioids, contemporary medical attention continues to focus primarily on the cure of the underlying disease and only minimally is directed to treating disease-associated pain. (Joint Commission) For a large number of patients, the undertreatment of post-operative pain delays out-of-bed patient mobilization, prolongs a patient's hospital stay, delays functional recovery, and/or increases monetary loss due to postoperative absences from work.

Typical side effects associated with the administration of morphine include: respiratory depression, constipation, and urinary retention. In some of the previous studies of epidural administration of morphine, these side effects have been reported. For example, Sepehrnia and van Ouwerkerk administered 10 mg of epidural morphine in subjects operated on for lumbar disc herniation, obtaining significant pain relief. (Sepehrnia A, van Ouwerkerk W J, “Analgesic effect of epidural morphine in lumbar disc surgery”, Neurosurg Rev 19: 227-230 (1996)) The authors reported that urinary retention was the relevant side effect.

Free morphine in the epidural space dilutes in the blood and tissue fluids and is absorbed systemically, remaining effective as an analgesic for 6-24 hours. (Bourke D L, et al., “Epidural opioids during laminectomy surgery for post-operative pain”, J Clin Anesth 4: 277-281 (1992); Rechtine G R, et al., “The use of epidural morphine to decrease postoperative pain in patients undergoing lumbar laminectomy”, J Bone Joint Surg (Am) 66: 113-116 (1984); Schnmidek H H & Cutler S G, “Epidural morphine for control of pain after spinal surgery. A preliminary report”, Neurosurg 13: 37-39 (1983)) Therefore, in order to allow the persistence of morphine in the epidural space, scientists have used different “vehicles” as drug depositories. For example, Gibbons used the absorbent properties of small pieces of gelatin sponge (10-mm thickness) injected with preservative-free morphine and methylprednisolone, contouring them to fit the laminectomy defect. (Gibbons K J, et al., “Lumbar discectomy: Use of an epidural morphine sponge for postoperative pain control”, Neurosurgery 36: 1131-1135 (1995)) Hurlbert used a microfibrillar collagen applied to the exposed dura before wound closure, mixed with methylprednisolone, morphine, and aminocaproic acid. (Hurlbert R J, et al., “A prospective randomized double blind controlled trial to evaluate the efficacy of an analgesic epidural paste following lumbar decompressive surgery”, J Neurosurg (Spine 2) 90: 191-197 (1999)).

Therefore it is an object of the invention to provide a method for decreasing the severity and time period for postoperative pain.

It is a further object of the invention to provide compositions for alleviating postoperative pain which do not induce harmful side effects.

### BRIEF SUMMARY OF THE INVENTION

A formulation containing an opioid such as morphine in a low dose in a controlled release carrier, such as the carbohydrate polymer gel marketed as ADCON®-L, can be administered to patients to treat pain over a period of one or more days following surgery of the spinal column or other structures associated with the central nervous system. In one embodiment, the morphine is administered at the end of a lumbar microdicectomy. A low dose can be used as compared to previous studies in which the morphine was administered in solution, typically at levels of about 2-4 mg morphine when administered in a paste, or 10 mg morphine when administered as an epidural solution.

### DETAILED DESCRIPTION OF THE INVENTION

I. Compositions

Compositions are formed of a carrier for controlled release of an opiod, optionally including other therapeutic, prophylactic or diagnostic agents, for example, an analgesic or antibiotic.

Carrier

A number of controlled release formulations are known for use in close proximity to or adjacent the spinal cord. These can be in the form of a polymeric or hydroxyapatite material such as a bone cement, microspheres, or gels.

Adhesion Control in a Barrier Gel (ADCON®-L) has been shown to act as a barrier to the development of epidural fibrosis following lumbar procedures, minimizing the formation of fibrotic scar and improving the long-term outcome. ADCON®-L is an agent applied frequently in Europe and in the United States to the epidural space at the end of spine procedures for lessening scar formation. It has been shown to be safe and effective in acting as a barrier to scar formation and surgical adhesions. (Brotchi J, et al., “Prevention of epidural fibrosis in a prospective series of 100 primary lumbo-sacral discectomy patients: follow-up and assessment at re-operation”, Neurol Res 21 Suppl 1: S47-50 (1999); de Tribolet N, et al., “Clinical assessment of a novel antiadhesion barrier gel: Prospective, randomized, multicenter, clinical trial of ADCON-L to inhibit postoperative peridural fibrosis and related symptoms after lumbar discectomy”, Am J Orthop 27:111-120, (1998); Frederickson R C, “ADCON-L: A review of its development, mechanism of action, and preclinical data”, Eur Spine J 5(Suppl 1): S7-9 (1996); McKinley D S, Shaffer L M, “Cost effectiveness evaluation of ADCON-L adhesion control gel in lumbar surgery”, Neurol Res 21 Suppl 1: S67-71 (1999); Robertson J T, et al., “Prevention of epidural fibrosis with ADCON-L in presence of a durotomy during lumbar disc surgery: Experiences with a pre-clinical model”, Neurol Res 21(Suppl 1): S61-66 (1999)) No adverse events or complications have been reported to date from its use. However, ADCON®-L has not been approved nor is it used as a drug depository.

ADCON®-L was employed as a vehicle for controlled drug delivery. Example 1 demonstrates that morphine is slowly released from ADCON®-L into the epidural space at the surgical site.

Analgesics

Preferred analgesics are opioids. The opioids may be natural or synthetic. Examples of natural opioids include morphine. Others include buprenorphine, Butorphanol, Codeine, Hydromorphone, Levorphanol, Meperidine, Methadone, Nalbuphine, Opium, Oxymorphone, and Pentazocine. Other analgesics may also be used.

Dosage

A low dosage of the analgesic is delivered to the patient to minimize side effects. In a preferred embodiment the dosage is less than 2 mg morphine total dosage. In the most preferred embodiment 1 mg of morphine is administered epidurally to the patient. As used herein, a low dosage is less than what has previously been administered in solution or in a carrier that does not provide controlled release. As used herein, controlled release means drug is released or provides pain control over a period of at least hours, more preferably at least one day, most preferably for two or more days.

Other Therapeutic, Diagnostic and Prophylactic Agents

Other agents may also be incorporated into the compositions. Examples include antibiotics, antiinflammatories, antivirals, chemotherapeutic agents, and growth factors.

II. Applications for the Compositions

The compositions can be administered to a site where pain control is needed by injection or direct implantion to the patient during surgery, such as a lumbar microdiscectomy.

### EXAMPLES

### Example 1

### Comparison of Epidural Administration of Morphine in ADCON®-L and ADCON®-L

The epidural administration of morphine dissolved in ADCON®-L employed at the end of spinal procedures provides immediate and prolonged relief of postoperative pain. As confirmed by the randomized double-blind placebo controlled trial on lumbar miscrodiscectomies described below, the use of morphine in a carrier, such as ADCON®-L, has no undesirable side-effects and is results in significant decreases in pain intensity, lower consumption of postoperative analgesics, shorter hospital stay, less postoperative work-time loss, and higher treatment satisfaction rate.

Clinical Material and Methods

A prospective, randomized, controlled double-blind study was conducted to evaluate the safety and efficacy of epidural morphine-ADCON®-L paste for pain relief after lumbar microdiscectomy. The study protocol received ethical and scientific approval from the hospital Investigational Review Board. Patients were eligible and enrolled in the trial if the following study inclusion criteria were fulfilled: (1) the patient's medical history and neurological examination were consistent with the diagnosis of lumbar radiculopathy, (2) neuroradiological evidence of lumbar disc herniation, with or without foraminal stenosis, existed, (3) a consistency between neurological examination, symptoms, and neuroradiological findings was determined, and (4) the patient had not undergone more than one previously performed lumbar microdiscectomy. Patients with the following medical histories were excluded from the study: (1) presence of spondylolistesis, (2) allergy or history of hypersensitivity to morphine, (3) history of dementia or severe cognitive impairment, and (4) history of clinically relevant hepatic, renal, and/or cardio-pulmonary insufficiencies or diseases. All patients signed informed consent forms before enrolling in the clinical trial.

One hundred patients were enrolled in the clinical trial. Enrolled patients were randomized to two groups: (1) 51 patients were in the morphine-ADCON®-L group (M-ADL) and (2) 49 patients were in the ADCON®-L control group (ADL). After the lumbar microdiscectomy, the patients were followed clinically over a period of 24 months.

Surgical Protocol

Before performing the surgical incision, 500 mg sulbactam and 1000 mg ampicillin were intravenously administered as antibiotic prophylaxis. All surgical procedures were performed via a standard midline microsurgical approach with dissection of the ligamentum flavum, followed by a minimal removal of bone, including foraminotomy. After dura and nerve root(s) decompression and accurate hemostasis, randomized patients received either (1) 1 mg of morphine (L. Molteni & Co., Florence, Italy) (1 ml a solution containing 10 mg of morphine was diluted in 10 ml of normal saline), which was dissolved into bioabsorbable ADCON-L (Gliatech Inc, Cleveland, Ohio) (10 cc) or (2) 1 ml of normal saline added to ADCON-L.

The M-ADL gel and the ADL gel were prepared on a separate sterile table by an operating room nurse, according to the randomization table. The gels were then given to the surgeons, who remained blinded as to whether a patient was part of the active group or the control group.

During the first 24 hours post-surgery, patient vital signs were taken every hour and blood oxygen saturation was constantly monitored by pulse oxymetry. Approximately 6-8 hours after completion of the surgery, patients were encouraged to stand and walk with the aid of a nurse. In the immediate post-operative time, intravenous ketoralac at the dose of 30 mg was available for pain control on an “as needed” basis every 12 hours.

Patients were discharged when they could stand and walk comfortably and unassisted. At home, patients were allowed to take oral diclofenac 75 mg once a day, if needed. Following hospital disharge, patients were asked to record daily in a diary the following information: (1) answer “yes” or “no” to the daily question: “Have you used the pain medication prescribed to you to relieve the pain due to surgery and/or your back and leg pain for which you underwent surgery?” and (2) record the number of days lost from work after surgery.

Study patients were clinically followed for 12 months. Two weeks after surgery, patients were asked to return to the clinic and record the intensity of their radicular pain on the visual analog scale (VAS). Patients were then asked to return for follow-up visits at the end of the first, third, sixth, and twelfth months after the surgery. During the first month after surgery, patients were also weekly surveyed by phone calls to monitor pain intensity, changes in motor and sensory function, and onset of urinary complaints.

The intensity of spontaneous ongoing pain was measured by using VAS, based on a 100 mm line, where the left-end of the line corresponds with “no pain” and the right-end to “the worst pain imaginable”. (Scott J, Huskisson E C, “Graphic representation of pain”, Pain 2:175-184 (1976)) Patients were asked to mark the VAS line at a point that they felt corresponded most accurately to their level of pain intensity. In particular, the patients were asked to record their VAS level of spontaneous low back and radicular pain at the following time intervals: (1) baseline before admission, (2) time of patient hospital disharge, and (3) first follow-up visit, i.e. two weeks after surgery.

During the same intervals, each patient underwent the straight-leg-raising (SLR) maneuver. SLR evoked pain was measured in degrees of straight leg angulation. For example, with the patient supine, the minimum degree of leg angulation is 0°, i.e. with the symptomatic leg fully extended and parallel to the examining table, while the maximum degree is 90°, i.e. with the leg fully extended and perpendicular to the table. In each patient, the maximum angle of straight leg elevation was calculated in relation to the maximum tolerated SLR-evoked pain, i.e. just before intolerable sciatic pain occurred.

At the two-week postoperative check-up, patient treatment satisfaction was measured using a four-grade scale: poor, mild, good, and excellent.

Results

Both groups were similar with respect to demographic data (Table 1). The mean duration in months of the patients' clinical history was 9.55±1.40 months in the M-ADL group and 8.78±0.99 months in the control group. The mean pre-operative (baseline) value in degrees of maximum straight leg elevation (i.e., angulation), before unbearable sciatic pain occurred on the SLR manuveur, was 37.94°±1.75 in the M-ADL group and 39.49°±2.17 in the control group. The baseline mean value of VAS pain intensity was 75.9 mm±13.9 in the M-ADL group and 76.3 mm±9.7 in the control group.

TABLE 1
Demographic and clinical data of M-ADL and ADL groups*

| | M-ADL group | ADL group |
|---|---|---|
| | (51 patients) | (49 patients) |
| Age (years) | 44.20 ± 1.70 | 44.29 ± 1.63 |
| Gender | 35 M, 16 F | 37 M, 12 F |
| Length of clinical | 9.55 ± 1.40 | 8.78 ± 0.99 |
| history (months) | | |
| Pre-operative SLR | 37.94 ± 1.75 | 39.49 ± 2.17 |
| (degrees) | | |
| Value of pre-operative | 75.9 ± 13.9 | 76.3 ± 9.7 |
| pain by VAS (mm) | | |
| Presence of inferior | 8 | 19 |
| limbs motor deficit | | |
| (# of patients) | | |
| Previous disc operation | 3 | 3 |
| at same level | | |
| (# of patients) | | |
| Level of disc herniation | | |
| (# of patients) | | |
| L2-L3 | 1 | 2 |
| L3-L4 | 4 | 3 |
| L4-L5 | 16 | 15 |
| L4-L5-S1 (two | 0 | 2 |
| interspaces) | | |
| L5-L6 | 0 | 1 |
| L5-S1 | 28 | 27 |
| Side of disc herniation | 34 R, 17L | 26 R, 23 L |
| (# of patients) | | |

*Mean values ±SE. Differences are not statistically significant Legend of Table 1: M = males; F = females; SLR = straight leg raising test; VAS = visual analog scale; R = right; L = left

Table 2 summarizes the postoperative outcome data. Forty-five patients of the M-ADL group were ambulatory within 6-8 hours of the completion of the procedure, whereas only three patients in the control group were comfortable with ambulation at that time (p<0.0001). All of the remaining patients in both groups were ambulatory 24 hours after the surgery. Patients felt fit and comfortable to leave the hospital at a mean postoperative discharge time of 1.37±0.07 days for the M-ADL group and 2.53±0.12 days for the control group (p<0.0001). In particular, 32 (63%) patients in the M-ADL group felt comfortable and were discharged home within 24 hours of the surgery. The remaining 19 (37%) patients of the M-ADL group were discharged on the second post-operative day. Only 2 (4%) patients of the control group felt well and ready for discharge within 24 hours of the surgery. Of the remaining patients in the control group, 27 (55%) patients left the hospital on the second day, 13 (27%) on the third day, and 7 (14%) on the fourth day after surgery. At 24 hours after surgery, the mean value in degrees of leg elevation on the SLR maneuver was 64.41°±1.59° in the M-ADL group and 57.77°±1.85° in the control group (p=0.02). The mean differences between the pre- and postoperative average values of SLR degrees were 26.47°±1.76° for the M-ADL group and 19.28°±1.80° for the control group (p=0.005).

At the same time, the mean postoperative pain intensity VAS value was 12.3 mm±0.9 in the M-ADL group and 24.7 mm±11.5 in the control group (p<0.0001). The mean differences between the pre- and postoperative average pain scores were 63.5 mm±2.0 mm for the M-ADL group and 51.6 mm±1.8 mm for the control group (p<0.0001). The mean 2-week postoperative VAS values of pain intensity were 7.4 mm±1.2 mm for the M-ADL group and 14.7 mm±0.9 mm for the control group (p<0.0001).

Hospital use of analgesics was recorded by nursing staff, who were involved in the care of the patients but were blinded regarding the placement of the patients in a treatment group. Within the first 24 hours after surgery, 27 (57%) patients in the M-ADL group and 47 (96%) patients in the control group required one or two doses of IV ketoralac (P<0.0001). As recorded by the same nursing staff at the two-week postoperative follow-up, 12 (23.5%) patients in the M-ADL group and 27 (55.1%) patients in the control group required almost one dose of oral diclofenac at home (p<0.0001).

At the two-week follow-up visit, treatment satisfaction was rated as “good” by 10 (20%) patients and “excellent” by 41 (80%) patients of the active group. In contrast, treatment satisfaction was rated as “good” by 20 (41%) patients and “excellent” by 29 (59%) patients of the control group (p=0.02). Finally, the mean postoperative work time loss was 21.67±0.92 days in the M-ADL group and 29.47±1.18 days in the control group (<0.0001).

TABLE 2
Postoperative data of M-ADL and ADL groups*

| | M-ADL group | ADL group | |
|---|---|---|---|
| | (51 patients) | (49 patients) | P |
| Intra-operative complications | none | none | |
| (# of patients) | | | |
| Post-operative complications (# | none | none | |
| of patients) | | | |
| Post-operative SLR (degrees) | 64.41 ± 1.59 | 57.77° ± 1.85 | 0.02 |
| Value of post-operative pain by | 12.3 ± 0.9 | 24.7 mm ± 11.5 | <0.0001 |
| VAS (mm) | | | |
| Patients ambulatory by the day | 45 | 3 | <0.0001 |
| of surgery | | | |
| (# of patients) | | | |
| Average day of discharge | 1.37 ± 0.07 | 2.53 ± 0.12 | <0.0001 |
| Assumption of post-operative | 57.4% | 95.9% | <0.0001 |
| analgesics | | | |
| (% of patients in group) | | | |
| Assumption of analgesics at | 23.5% | 55.1% | <0.0001 |
| home (2-week-f/up) (% of | | | |
| patients in group) | | | |
| Two-week subjective overall | E 80.4%, G 19.6% | E 59.2%, G 40.8% | 0.02 |
| result | | | |
| (% of patients in group) | | | |
| Mean post-operative work time | 21.67 ± 0.92 | 29.47 ± 1.18 | <0.0001 |
| loss (days) | | | |

*Mean values ±SE. Legend of figure: P = level of significance; SLR = straight leg raising test; VAS = visual analog scale; f/up = follow-up; E = excellent; G = good

No intraoperative nor postoperative complications were observed in either group. Nor were episodes of urinary retention, respiratory disturbances, or wound infections observed. Lumbar MRI and CT scans performed in patients 6-12 months after surgery confirmed that no relevant peridural fibrosis had developed in the M-ADL group of patients. Moreover, in both groups, at the one-year follow-up, there was no clinical evidence of late onset neurological complications, such as radiculopathies associated with peridural fibrosis or arachnoiditis.

1. A composition for treating operative and postoperative pain comprising a controlled release carrier and an opioid, wherein the carrier minimizes fibrotic scar formation and wherein an effective amount of opioid is released in a space to be treated, such as an epidural space, to provide pain control over a period of at least hours.

2. The composition of claim 1 wherein the pain control last for at least one day.

3. The composition of claim 1, wherein the carrier is selected from the group consisting of gels, microparticles, and implants.

4. The composition of claim 3 wherein the implants are formed of the polymer.

5. The composition of claim 3 wherein the gel is a carbohydrate gel.

6. The composition of claim 1, wherein the opioid is morphine.

7. The composition of claim 1 further comprising a therapeutic, diagnostic or prophylactic agent.

8. The composition of claim 7 wherein the agent is selected from the group consisting of antibiotics, antiinflammatories, antivirals, chemotherapeutic agents, and growth factors.

9. A method for reducing postoperative and operative pain comprising
administering to patient a composition comprising a controlled release carrier and an opioid, wherein the carrier minimizes fibrotic scar formation and wherein an effective amount of opioid is released in a space to be treated, such as an epidural space, to provide pain control over a period of at least hours.

10. The method of claim 9, wherein the composition is administered by injection.

11. The method of claim 9 wherein the composition is administered during a surgical procedure.

12. The method of claim 11, wherein the operation is a microdiscectomy.
